(19) United States
(12) Patent Application Publication
(10) Pub. No.: US 2009/0149746 A1
(43) Pub. Date: Jun. 11, 2009

(54) POST-BIOPSY CAVITY TREATMENT IMPLANTS AND METHODS

(75) Inventors: Ary CHERNOMORSKY, Walnut Creek, CA (US); John Soo Hoo, Union City, CA (US); James W. Vetter, Portola Valley, CA (US)

Correspondence Address: YOUNG LAW FIRM, P.C.;ALAN W. YOUNG, 4370 ALPINE ROAD, SUITE 106, PORTOLA VALLEY, CA, 94028, US

(73) Assignee: RUBICOR MEDICAL, INC., Redwood City, CA (US)

(21) Appl. No.: 12/273,754

(22) Filed: Nov. 19, 2008

Related U.S. Application Data
(60) Provisional application No. 60/989,064, filed on Nov. 19, 2007.

(51) Int. Cl. A61M 25/00; A61B 6/00

(52) U.S. Cl. 600/433; 604/518; 604/82

(57) ABSTRACT

A method of forming a soft tissue biopsy cavity marker may include steps of providing a radio-opaque element and a bio-compatible and bio-degradable polymer such as alginate and delivering the provided radio-opaque element and the provided polymer to a biopsy site, such as a breast biopsy site. A gelling initiator that includes divalent cations such as NaCl₂ may then be provided and delivered to the biopsy site, causing the previously delivered polymer to gel and to form a soft tissue biopsy marker in situ (within the biopsy site).

### BACKGROUND OF THE INVENTION

### Field of the Invention

The present invention relates to the treatment of soft tissue cavities formed as a result of biopsies or therapeutic procedures.

### SUMMARY

An embodiment of the present inventions is a soft tissue biopsy cavity marker formed in situ (that is formed within the patient's body, for example).

Another embodiment of the present inventions is a method, comprising providing a polymer in fluid form; delivering the provided polymer to a biopsy site; providing a gelling initiator that is configured to cause the polymer to gel, and delivering the provided gelling initiator to the biopsy site.

A step may be carried out of providing a radio-opaque element and wherein the first delivering step also delivers the provided radio-opaque element to the biopsy site. The first and second delivering steps may be carried out such that the polymer and the gelling initiator are initially separated from one another and only come into contact with one another within the biopsy site to form a soft tissue biopsy marker in situ. The first providing step may be carried out with the polymer being bio-compatible and bio-degradable. The first providing step may be carried out with the polymer including alginate. The second providing step may be carried out with the gelling initiator including divalent cations. The polymer delivering step may be carried out before the gelling initiator delivering step. The gelling initiator delivering step may be carried out before the polymer delivering step. The first providing step may be carried out with the alginate being dispersed in an aqueous solution at a concentration of, for example, about 0.1% to about 30% by weight. The first delivering step may deliver about 0.01 cc to about 400 cc of the polymer to the biopsy site and the second delivering step may deliver about 0.01 cc to about 800 cc of the initiator to the biopsy site. The radio-opaque element providing steps may be carried out with the radio-opaque element having a shape that is configured to facilitate the radio-opaque element being entrained with the polymer during the first delivering step. The radio-opaque element may have, for example, a generally cylindrical or helical shape having a first end and a second end, at least the first end being tapered.

Yet another embodiment of the present inventions is a device, comprising a first source of polymer; a second source, separate from the first source, of a gelling initiator that may be configured to gel the polymer, and a catheter configured to deliver the polymer and the gelling initiator to a patient.

The catheter may define a single lumen. Alternatively, the catheter may define a first lumen coupled to the first source of the polymer and a second lumen coupled to the second source of the gelling initiator. The device may further include a radio-opaque element disposed within the first lumen of the dual lumen catheter. The polymer may include alginate. The gelling initiator may include divalent cations. The catheter may be configured to deliver the polymer and the gelling initiator separately to the biopsy site. The catheter may define an internal lumen and the first source of the polymer may be the internal lumen of the catheter. The device may further include an elongate piston configured to engage and slide within the internal lumen and to push the polymer out of the internal lumen. The piston and the catheter may define respective distal tips and the distal tip of the piston may be proximal to the distal tip of the catheter when the piston is fully engaged within the catheter. At least portions of both the catheter and the piston may be configured with selective rigidity and/or flexibility.

A still further embodiment of the present inventions is a kit, comprising a device, comprising a catheter that may be pre-loaded with a bio-compatible and bio-degradable polymer; a source of a gelling initiator that may be configured to gel the polymer, the source of gelling initiator being configured to be coupled to the catheter, and a radio-opaque element, and sterile packaging encapsulating the device. The device may be formed of plastic materials and may be configured for single use.

Yet another embodiment of the present inventions is a breast biopsy marker formed of a gelled polymer, the marker including a bulbous body portion and a tail portion extending from the bulbous body portion.

### BRIEF DESCRIPTION OF THE DRAWINGS

For an understanding of the objects and advantages of the present invention, reference should be made to the following detailed description, taken in conjunction with the accompanying figures, in which:

FIG. 1 shows a device according to an embodiment of the present invention, in a first state in which a distal portion of the device has been inserted into a soft tissue cavity.

FIG. 2A shows the device of FIG. 1, in a second state in which a bio-degradable polymer has been delivered to the soft tissue cavity.

FIG. 2B shows a portion of the device of FIG. 1, showing the radio-opaque element partially entrapped within the polymer delivered to the patient.

FIG. 3 shows the device of FIG. 1 in a third state, in which a gelling initiator has been delivered to the soft tissue cavity to at least partially immerse the polymer therein to form the soft tissue biopsy marker in situ, according to an embodiment of the present invention.

FIG. 4 shows the device of FIG. 1 as it is being removed from the soft tissue cavity, leaving the in situ-formed soft tissue biopsy marker within the cavity.

FIG. 5 shows the device of FIG. 1 being used in conjunction with another biopsy device.

FIG. 6 is a flowchart detailing steps for forming a soft tissue cavity marker in sit, according to an embodiment of the present invention.

FIG. 7A shows a radio-opaque element that may form an integral part of the present soft tissue biopsy marker, according to an embodiment of the present invention.

FIG. 7B shows another radio-opaque element that may form an integral part of the present soft tissue biopsy marker, according to another embodiment of the present invention.

FIG. 8 shows another embodiment of the present invention configured to spread soft tissue in the vicinity of the distal tip of the device to allow for the formation of a soft tissue cavity marker in situ, in the absence of a pre-existing cavity.

FIG. 9 shows another embodiment of the present inventions.

FIG. 10 shows yet another embodiment of the present inventions.

FIG. 11 shows a kit, according to still another embodiment of the present inventions.

FIG. 12 shows another embodiment of the present invention.

FIG. 13 shows the embodiment of FIG. 12, after delivery of the polymer to the cavity.

FIG. 14 shows the embodiment of FIG. 12, after both the polymer and the initiator have been delivered to the cavity.

FIG. 15A shows the embodiment of FIG. 12 being retracted from the patient's tissue.

FIG. 15B is a partial view of an embodiment of the present invention that is configured to form all in situ marker without a tail.

FIG. 15C shows a partial view of an embodiment of the present invention that is configured to form an in situ marker with a tail of length d₁.

FIG. 15D shows a partial view of an embodiment of the present invention that is configured to form an in situ marker having a tail of length d₂.

### DESCRIPTION OF EMBODIMENTS OF THE INVENTION

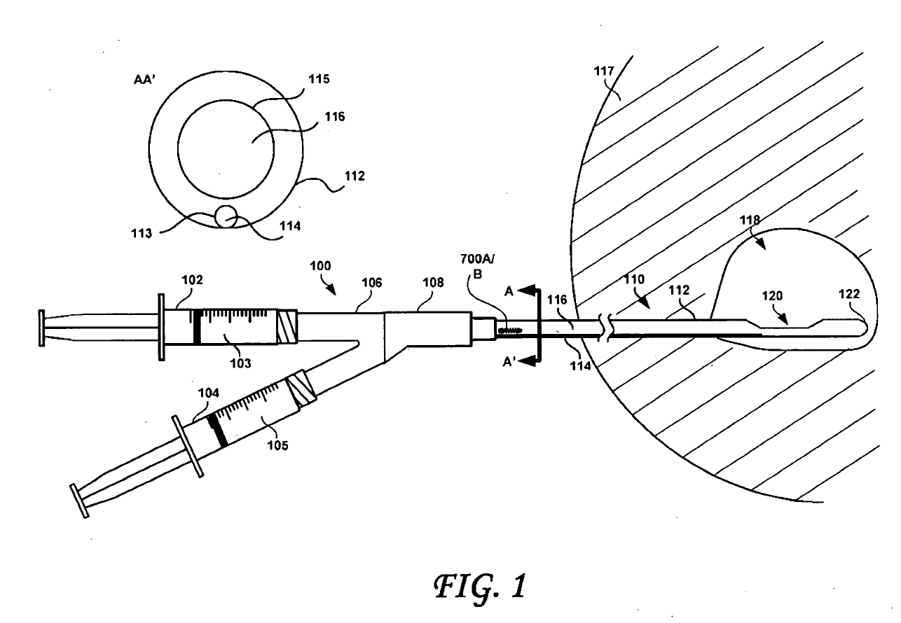

FIG. 1 shows a view of a device 100 according to an embodiment of the present invention, in a first state in which a distal portion of the device has been inserted into a soft tissue cavity 118. As shown therein, the device 100 includes a first source 102 of a polymer 103 and a second source 104 of an initiator or catalyst 105. According to embodiments of the present invention, the polymer 103 may include a polymer-containing liquid that is susceptible to gelling (becoming a gel) when subjected to certain environmental conditions. For example, the polymer may include a solution containing an effective amount of a bio-compatible and bio-degradable natural polymer, such as alginate. Alginate, extracted from seaweed, is a linear copolymer that may include homopolymeric blocks of (1-4)-linked β-D-mannuronate (M) and its C-5 epimer α-L-guluronate (G) residues, respectively, covalently linked together in different sequences or blocks. The monomers may appear in homopolymeric blocks of consecutive G-residues (G-blocks), consecutive M-residues (M-blocks), alternating M and G-residues (MG-blocks) or randomly organized blocks. The alginate may be dispersed in a solvent (such as an aqueous solution, for example). The amount of alginate dispersed in the aqueous solution may be freely chosen, subject to the constraints that the alginate solution 103 should have a sufficiently low viscosity to be injectable or otherwise delivered to the patient's tissue and should have an effective amount of alginate to form a sufficiently firm gel so as to function as a proper marker. Practically, the concentration of alginate in the aqueous solution may be as low as possible, as long as a sufficiently firm gel is formed in situ, with the understanding that higher concentrations yield firmer gels. For example, a concentration of alginate in the aqueous solution may be selected within the range of about 0.1% to about 30% by weight. For example, a concentration of alginate in the aqueous solution may be selected within the range of about 1% to about 20% by weight. For example, a salt of alginic acid (such as, for example, the sodium salt of alginic acid NaC₆H₇O₆) may be used as the polymer 103. The in situ formed marker should be sufficiently firm so as to substantially immobilize a radio-opaque element, as described fully below.

Alginate gels can develop and set at constant temperature. However, the polymer 103, while capable of gelling and creating a polymer network or matrix acting as a tissue marker, will not do so in the absence of a gelling initiator or catalyst. Such a gelling initiator or catalyst is shown in FIG. 1 at 105 and is contained in a separate second source, referenced at numeral 104. The second source may include, as may the first source 102, a syringe. It is noteworthy that the first and second sources, according to this embodiment, are separate and distinct from one another, and are configured to keep the polymer 103 and the initiator 105 out of contact with one another. Were the polymer 103 and the initiator not kept apart, the polymer 103 could prematurely react with the gelling initiator mid could prematurely (i.e., before delivery to the patient) solidify as a gel, which could prevent the gelled polymer from being injected or otherwise readily delivered to the cavity. An alginate gel may be considered part solid and part solution. After gelation, the water molecules are physically entrapped by the alginate matrix, but are still free to migrate. Alginate gel develops in the presence of a divalent ionic solution that includes, for example, cations such as Ca²⁺, Br²⁺ or Sr²⁺, for example. Here, a calcium salt with good, or limited solubility, or complexed Ca²⁺ ions may be mixed with an alginate solution into which the calcium ions are released. According to an embodiment of the present invention, the gelling initiator may be or include CaCl₂ and the polymer 301 may be or include a sodium alginate solution (such as, for example, the PRONOVA™ material manufactured by FMC Biopolymers of Philadelphia, Pa., d/b/a NovaMatrix). Using the polymer 103 and the initiator 105, an alginate solution can be solidified by an internal gelation/setting method, i.e. in situ (within the patient's tissue 117, such as within the breast). As for relative quantities of polymer vs. initiator, the first source 102 may be configured to contain, for example, about 0.01 cc to about 350 cc of polymer. For example, the first source 102 may be configured to contain 0.1 cc to about 3.5 cc of polymer 103 (such as the above described alginate-containing solution) and the second source 104 may be configured to contain about 0.01 cc to about 700 cc of initiator. For example, the second source 104 may be configured to contain about 0.1 cc to about 7 cc of initiator (such NaCl₂), for example. However, it is to be understood that the above ranges are only exemplary in nature and that other ranges may be effective depending upon the application, as those of skill in this art may appreciate.

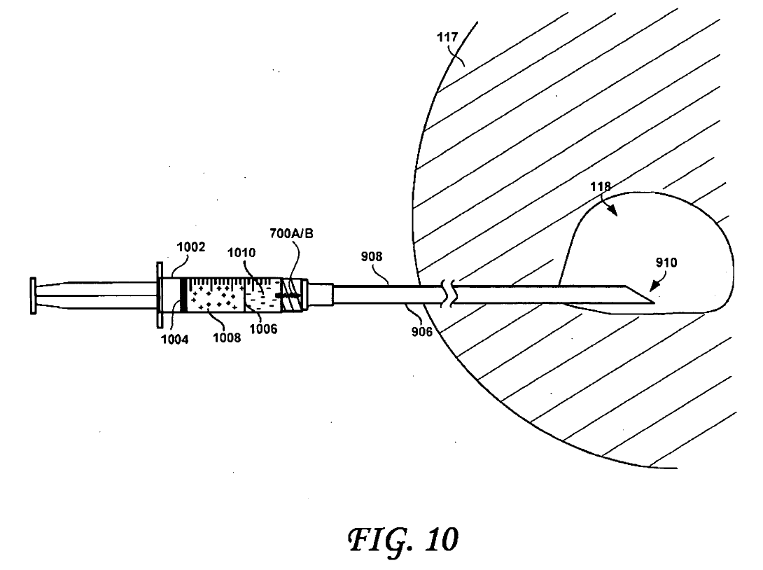

Tuning back to FIG. 1, the first source 102 of die polymer 103 may be a syringe, as shown in FIG. 1, as may be the second source 104 of the initiator 105. Note, however, that the first and second sources may be combined in a single delivery device, such as die dual-chambered syringe shown in FIG. 10, for example. The first and second sources 102 and 104 may be coupled to a “Y” tubing member 106 which is itself coupled to a dual lumen catheter 112 through a connector 108. According to all embodiment of the present invention, the dual lumen catheter 112 may include a outer surface that is configured to come into contact with the patient's tissue 117, a first internal lumen 116 formed by a first internal surface 115 within the dual lumen catheter 112 and a second lumen 114 formed by a second internal surface 113 within the dual lumen catheter. According to an embodiment of the present invention, the first lumen 116 may be coupled to the first source 102 of the polymer 103 and the second lumen 114 may be coupled to the second source 104 of the gelling initiator 105. The first lumen 116 of the dual lumen catheter 112 may have a diameter that is significantly larger than the diameter of the second lumen 114 of the dual lumen catheter 112, to allow for the efficient passage of the substantially more viscous polymer 103 (as compared to the viscosity of the initiator 105) through the catheter 112. The first and second lumen 116, 114 are configured to keep the polymer 103 and the initiator 100 separate from one another until each reaches the distal portion of the device, at or adjacent to the distal tip 122 of the device 100.

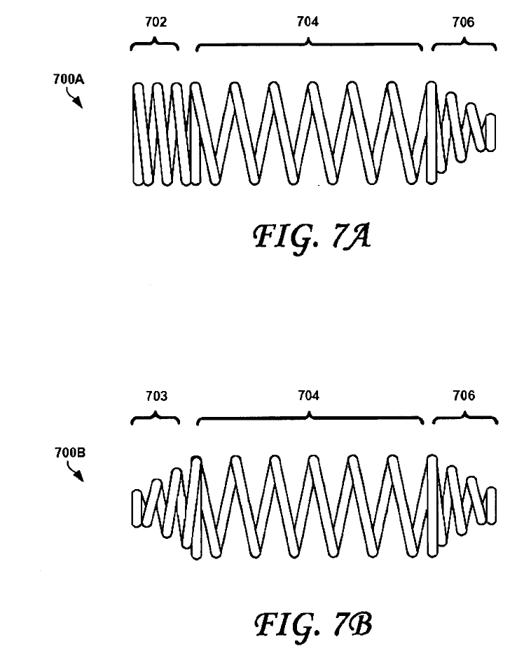

Also as shown in FIG. 1, the device 100 may be configured to deliver a radio-opaque (that is, visible under X-ray and/or ultrasound, for example) element (which may, but need not be, configured as shown at 700A and 700B in FIGS. 7A and 7B) to the biopsy site. For example, the radio-opaque element may be formed of a bio-compatible metal such as, for example, stainless steel, or titanium, or Nitinol®, a nickel-titanium alloy. The radio-opaque element 700A, 700B may have most any shape, although the shapes shown in FIGS. 7A and 7B are believed to be advantageous, as such shapes are readily visible and appears clearly artificial under most imaging modalities, whatever their orientation within the cavity 118. Yet another advantage of the above-described shapes their susceptibility to becoming entrained within the polymer being pushed toward the cavity, even when the polymer does not exhibit a great degree of viscosity.

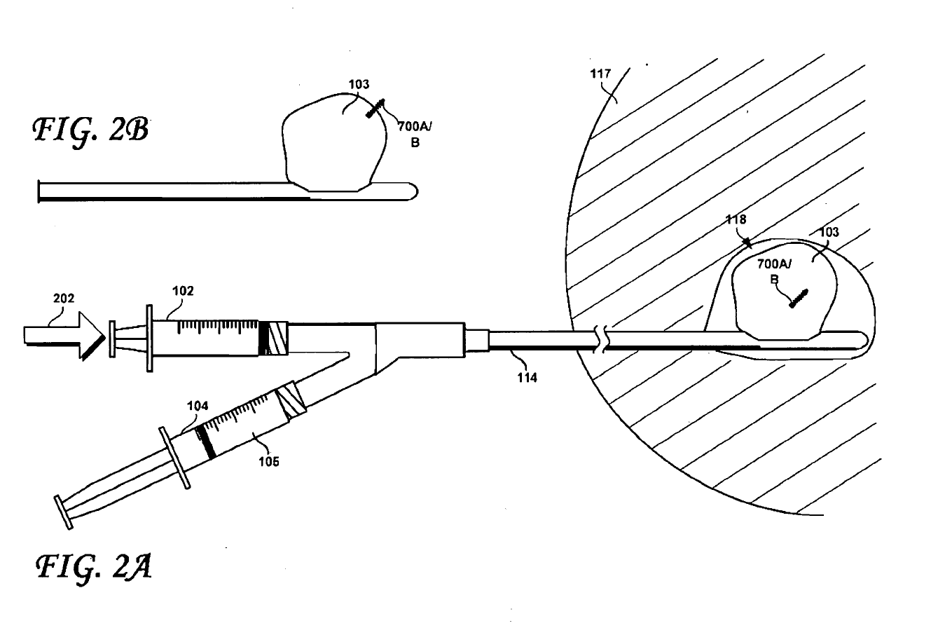

In use, the distal portion of the device 100 may be inserted into the patient's tissue 117 (such as the breast, for example), at least until the distal tip 122 of the device 100 is disposed within the cavity 118 where the marker according to embodiments of the present inventions is to be formed and disposed. FIG. 1 shows the device 100 with the distal portion thereof disposed within a cavity (e.g., a void or a volume from which a biopsy specimen has been cut and removed) 118 within the patient's tissue 117. Turning now to FIG. 2A, the device 100 is shown therein in a state in which the polymer 103 has been delivered (in this case, injected by pushing on the plunger of the syringe 102) to the cavity 118, through the opening 120. According to an embodiment of the present invention, the radio-opaque element 700A, 700B is entrained in the flow of the polymer 103 within the first lumen 116 and is delivered, along with a bolus of the polymer 103, within the cavity 118 formed in the patient's tissue 117 through a free end of the lumen at or adjacent the distal tip 122 of the device 100. The radio-opaque element 700A, 700B may be fully encapsulated within the bolus of polymer 103 (as shown in FIG. 2A) or may be merely partially contained within the bolus of polymer 103, with a portion of the radio-opaque element 700A, 700B protruding from the bolus of polymer 103, as shown in FIG. 2B. The polymer 103, in this state, is still a fluid, although it may be a more or less viscous fluid, depending upon the amount and chemical composition of alginate in the solution. Significantly, in the state shown in FIGS. 2A and 2B, the bolus of polymer 103 with the entrapped and/or partially contained radio-opaque element 700A, 700B is not an ideal functional biopsy cavity marker, as the bolus is not completely solid and cannot function to substantially immobilize the radio-opaque element within the cavity 118.

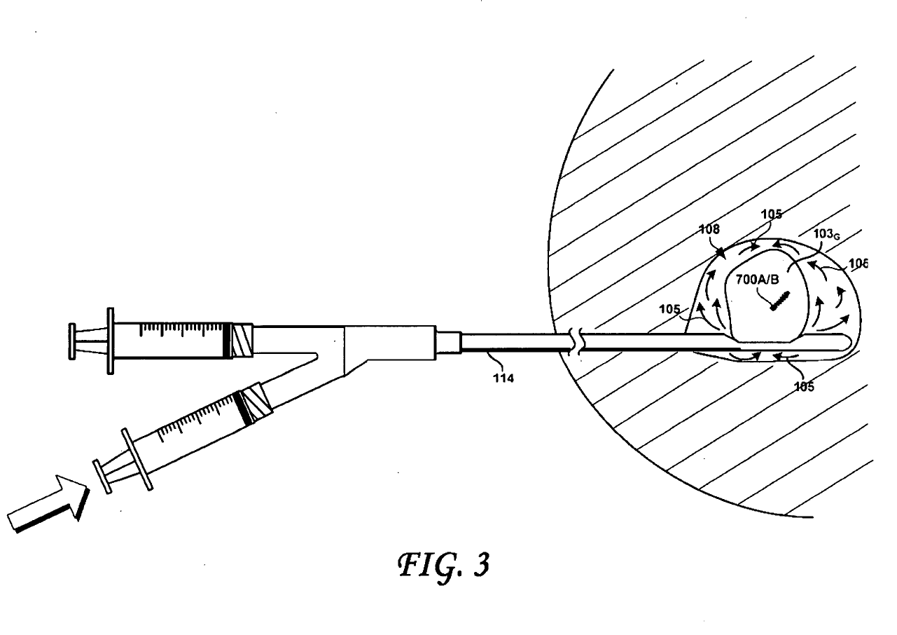

To complete the formation of the biopsy cavity marker in situ, according to embodiments of the present invention, requires the gelation of the bolus of polymer 103. Gellating the bolus of polymer may be carried out, according to embodiments of the present invention, by delivering a volume of the gelling initiator 105 to the cavity 118, where the previously delivered bolus of polymer and the radio-opaque element 700A, 700B are disposed. This may be done by delivering the gelling initiator to the cavity 118. This may be carried out by, in this embodiment, pushing down on the plunger of the syringe that functions as the second source 104 of the initiator 105, as shown in FIG. 3. This forces the gelling initiator (such as CaCl₂, for example) through the second lumen 114 of the dual-lumen catheter 112 to a free end thereof, disposed at or next to the distal tip 122 of the device 100. In so doing, the bolus of polymer 103 becomes at least partially immersed in the gelling initiator 105. The polymer 103 then reacts with the initiator 105 by becoming a gel 103_{G}, that is, an alginate-containing (in this embodiment) substance having the density similar to a fluid and the structural coherence of a solid, enabling it to substantially maintain a given shape. It should be noted that the above-described steps of creating a gelled marker in situ may be performed in reverse order: the initiator 105 may be delivered to the cavity first, followed by the delivery of the polymer 103 to the cavity. However created, the bio-compatibility and biodegradability of alginate gels, together with their density and structural characteristics, makes them ideal candidates for in situ tissue markers.

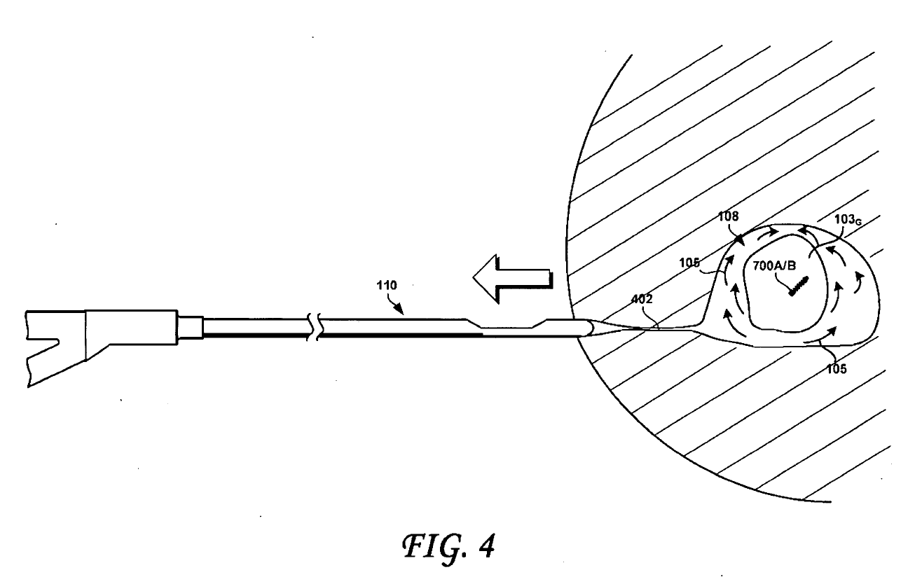

After delivery of the initiator 105, the device 100 may be left in place within the cavity 118 for a short period (on the order of a few seconds to about a minute, typically about 30 seconds), to allow for the gel 103_{G} to set. After the gel 103_{G} has solidified enough to allow the retraction of the distal portion of the device 100 from the cavity 118, the device 100 may be retracted, as shown in FIG. 4 along, for example, the retraction path 402 of the originally inserted biopsy instrument. Note that the newly in situ-formed soft tissue cavity marker will continue the gelation process and become increasingly firm for a short period of time, until a steady state is reached where the initiator 105 no longer has an appreciable gelation effect upon the polymer 103. Note that the soft tissue cavity marker (including the gelled polymer 103_{G} and the radio-opaque element 700A, 700B) does not exist prior to delivery thereof into the cavity 118. Indeed, prior to delivery to the cavity 118, only the constituent elements (including the radio-opaque element and the precursor elements 103 and 105) of the soft tissue cavity marker exist. It is only when the polymer 103 (containing the at least partially entrapped radio-opaque element 700A, 700B) is at least partially immersed in the delivered initiator 105 that the marker (now including the gelled polymer 103_{G} and the radio-opaque element) comes into being in situ. Outside of the body, there is no marker, only the constituent elements thereof, wherein the polymer is only in a precursor “un-gelled” form 103 (that is structurally and functionally different from the polymer 103), as compared to the gelled polymer 103_{G}.

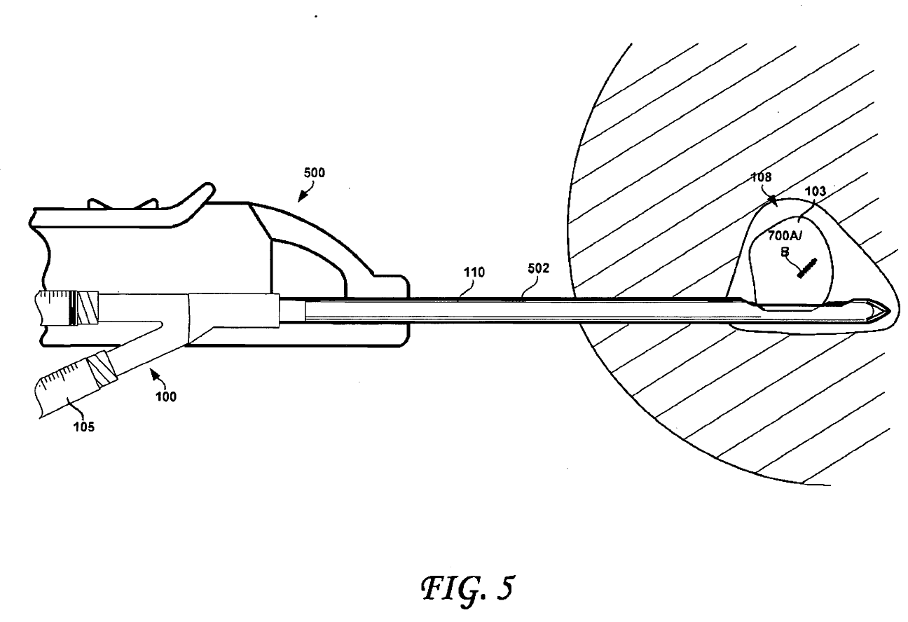

FIG. 5 shows the device of FIG. 1 being used in conjunction with another biopsy device. In the example illustrated in FIG. 5, the biopsy device is a Mammotome® biopsy system 500, marketed by Johnson & Johnson Ethicon Endo-Surgery. Indeed, the dual lumen catheter of the device 100 may be inserted directly into the tissue collecting shaft 502 of the Mammotome® biopsy system 500, such that its distal end through which the polymer 103 and the initiator 105 are delivered lines up with the opening at the distal working end of the Mammotome® biopsy system 500. In this manner, precise emplacement of the device 100 may be assured, as the dual lumen catheter 110 of the device 100 is axially engaged into the shaft 110 of the Mammotome® biopsy system 500 and as the distal portion of the present device 100 is precisely guided to the cavity 118 by the very instrument 500 that created the cavity 118. Therefore, the physician may guide the present device 100 to a precise location within the patient's tissue (under ultrasonic guidance, for example), and may form a soft tissue cavity marker in situ, all before removing the biopsy instrument, such as the Mammotome® biopsy system 500. Indeed, after waiting for a short period of time (e.g., 30 seconds or less, depending, for example, upon the formulation of the polymer 103 and the initiator 105), the physician may then remove the Mammotome® biopsy system 500 together with the present device 100 still engaged therein, and close the incision. The Mammotome® biopsy system forms no part of the present inventions and is shown and discussed herein for exemplary purposes only, it being understood that the embodiments shown herein are not limited thereto.

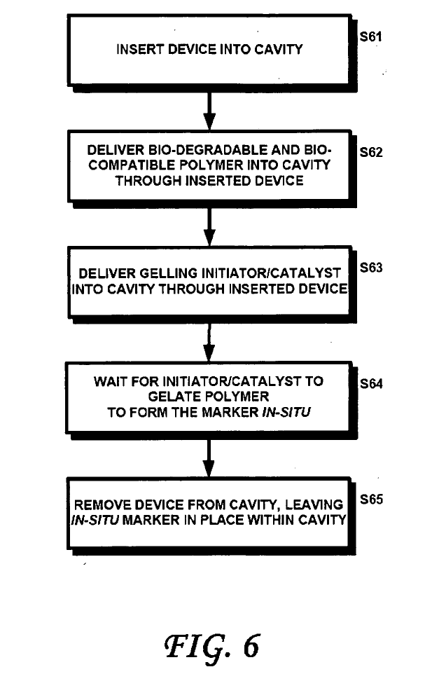

FIG. 6 is a flowchart detailing steps for forming a soft tissue cavity marker in situ, according to an embodiment of the present invention. As shown therein, step S61 calls for the distal portion of the device 100 to be inserted into a preformed cavity 118. As will be detailed below, embodiments of the present inventions may be adapted for use in cases wherein no cavity is present. In such a case, step S61 would call for inserting the device 100 into the patient's tissue. Next, step S62 calls for delivering a suitable gelable biodegradable polymer 103 into the cavity through the inserted device 100. The device 100 may also include a radio-opaque element 700A or 700B pre-loaded therein, such that the radio-opaque element 100 is delivered (preferably but not necessarily concurrently) with the polymer 103. As noted above, the gellable bio-compatible polymer 103 is also bio-degradable. After the polymer 103 is delivered, the initiator or gelling catalyst 105 may be delivered, also through the inserted device 100, as shown at S63. This causes the gelation of the polymer 103, to form a soft tissue marker in situ, including the gelled polymer 103_{G} and an at least partially entrapped/encapsulated radio-opaque element 700A or 700B. The physician may then choose to wait for a short period of time, as shown at S64, to allow for the polymer 103 to at least begin the gelation process in the presence of the initiator 105. The device 100 may then be removed from the patient's tissue, leaving the in situ-formed marker in place within the cavity, as called for by step S65. As noted above, The process of in situ marker formation may be performed in reverse order, by delivering the initiator 105 before the polymer 103 is delivered.

FIG. 7A shows a side view of a radio-opaque element 700A suitable for use with the present soft tissue markers, methods of forming in situ tissue markers and devices for forming and delivery in situ tissue markers. As shown therein, one embodiment of the radio-opaque element 700A includes three portions; namely, a first portion 702, a second portion 704 and a third portion 706. The radio-opaque element 700A is preferably made of a single piece of radio-opaque material such as, for example, stainless steel, titanium, or Nitinol®. As shown, the radio-opaque element 700 may (but need not) be generally spring-shaped, and may include a first portion 702 having tightly wound coils, a second portion 702 having less tightly wound coils and a third portion 706 having tightly wound coils having progressively smaller diameters toward the free end of the radio-opaque element 700A, to give the radio-opaque element 700A a generally tapered shape resembling a bullet. Functionally, the shape of the radio-opaque element 700A is advantageous, as it is readily visible under ultrasound or X-ray, irrespective of the orientation of the element 700 relative to the active imaging plane of the ultrasound or X-ray device. Indeed, should the radio-opaque device 700A be imaged axially, the tapered end thereof formed by the tightly wound coils of progressively decreasing diameter of the third portion 706 will present as a clearly artificial construct within the tissue 117, as opposed to a faint ring (as a simple tube without a tapered end would appear when seen axially edge on). The shapes of the radio-opaque elements 700A, 700B facilitate the elements being carried to the cavity by polymers 103 having even a low viscosity.

FIG. 7B shows another embodiment of a radio-opaque element 700B in which both free ends are tapered. Indeed, the radio-opaque element 700B includes a first portion 703 having tightly wound coils of progressively larger diameters, a second portion 704 of less tightly wound coils having a constant diameter and a third portion 706 of tightly wound coils having progressively decreasing diameters. Other implementations are possible, as those of skill in this all may appreciate. That is, embodiments of the present invention are not limited to the shape or structure of the radio-opaque element.

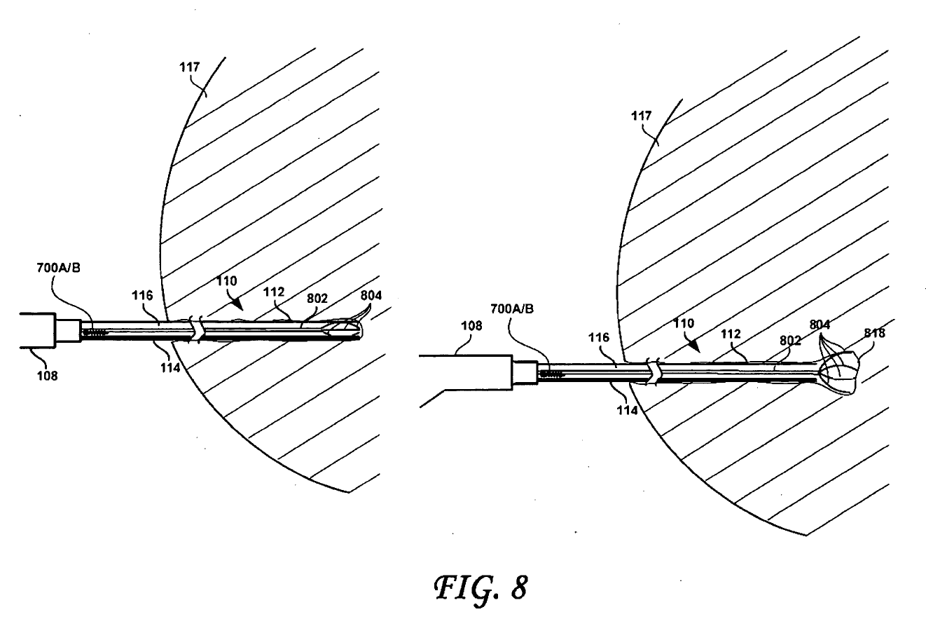

FIG. 8 shows an exemplary embodiment of a device 100 that may be used to form an in situ tissue marker as shown herein and described above, in the absence of a pre-formed cavity within the patient's tissue 117. As shown, the device 100 is similar to that shown in FIGS. 1-5. However, the device shown in FIG. 8 may include strictures that are effective to spread tissue at the distal end of the device 100 to make room for the tissue marker to be formed therein. According to one embodiment, a tissue spreader that is effective for that purpose may include an axial shaft 802 disposed within the larger first lumen 116 of the dual lumen catheter 112. A mechanism may also be provided at the distal end of the axial shaft 802 to spread tissue when the tissue spreader is deployed. For example, the tissue spreader may include a plurality of outwardly-biased curved spring-like wire or ribbon members 804 that fit, in a first compressed configuration, within the first lumen 116 of the dual catheter lumen 112. The distal tip of the device may then be positioned adjacent where the in situ marker is to be formed and the axial shaft 802 may then be pushed (by a suitable actuator disposed proximally oil the device 106) in the distal direction until the members 804 emerge from the first lumen 116 and into the mass of tissue 117. The members 804, released from the confines of the first lumen 116, will then decompress and expand axially like the petals of a flower, to thereby correspondingly compress the surrounding tissue 117 to create a small void, space or cavity 818 into which the present tissue marker may be formed in situ. Other embodiments for spreading tissue and creating space for a tissue marker to be formed in situ may well occur to those of skill in this art. All such alternate implementations are deemed to fall within the scope of the present inventions.

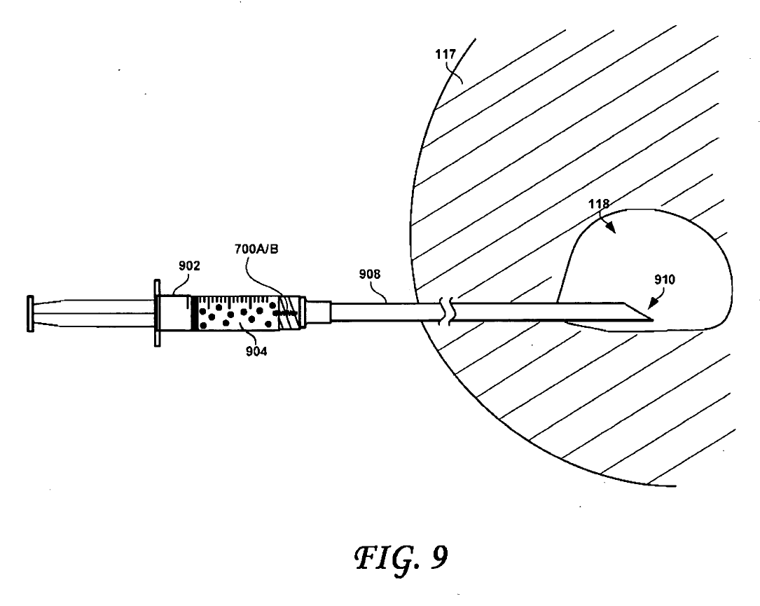

FIG. 9 shows another embodiment of the present inventions. As shown therein, a single source 902, in the form of a single syringe, may contain an injectable solution 904 that may be configured to form a polymeric gel in situ. For example, the injectable solution 904 may be a mixture of the polymer 103 and one or more initiator agents that collectively form the initiator 105. The injectable solution 904, moreover, may be configured to maintain its liquid form for a predetermined period of time, to allow for the solution 904 to be delivered to the patient prior to gelation. For example, the injectable (or otherwise deliverable) solution 904 may include a mixture of hydrochloric acid (HCl), calcium carbonate (CaCO₃) and soluble alginate. In such a mixture, the calcium carbonate reacts with the hydrochloric acid to form calcium chloride (CaCl₂). In turn, the calcium ions of the calcium chloride are complexed with the soluble alginate to form an aqueous insoluble calcium alginate gel. The water-soluble alginate may include, for example, ammonium, potassium, magnesium and sodium salts of alginate or mixtures of one or more of these. As long the physician does not wait too long, the solution 904 may be delivered to a cavity 118 before the calcium alginate gels to a degree that would render injection difficult, to thereby form the tissue marker in situ. As the solution 904 is pre-mixed, only a single syringe 902 may be necessary and only a single-lumen catheter 908 need be coupled thereto for delivery of the solution 904 to the cavity 118. The single-lumen catheter 908 may, for example, have a beveled distal tip 910, as shown in FIG. 9. Such an embodiment may be sold with the polymer 103 and the one or more gelling initiator agents (e.g., HCl and CaCO₃) packaged separately in pre-measured quantities, with the physician mixing them together before injection into the cavity 118. The relative quantities of the polymer and the gelling initiators in the solution 904 may be selected so as to delay gelation to allow for the mixture to be readily delivered to the cavity.

As shown at 700A/B, the radio-opaque element need not be, as shown in FIG. 1, be pre-loaded into the catheter 112. Instead, the radio-opaque element 700A/B (or other radio-opaque element) may instead be pre-loaded within the single syringe 902. Specifically, the radio-opaque element 700A/B (or other radio-opaque element) may be disposed, prior to delivery to the patient, in the luer lock of the single syringe 902, as shown in FIG. 9. The radio-opaque element should be maintained in place by the viscous solution 904 and should be pushed into and through the catheter 908 when the physician pushes in the plunger of the syringe to form the tissue marker in situ.

Alternatively, and as shown in FIG. 10, a single source may be used to sequentially deliver the polymer 103 and the initiator 105. As shown in FIG. 10, a dual-chambered syringe 1002 may be used to sequentially deliver pre-measured quantities of the polymer 103 and the initiator and/or initiator agents. An advantage of such dual-chambered syringes is that the polymer 1010 and the initiator and/or initiator agents 1008 may be kept separate prior to and until they are sequentially pushed into the cavity 118. As shown in FIG. 10, when the physician pushes down on the plunger 1004, the impermeable barrier separating the two chambers will be breached and the polymer 1010 and the radio-opaque element 700A/B will first be injected into the cavity, followed immediately by the initiator and/or initiator agents 1008, which will cause the gelation in site of the delivered polymer within the patient's tissue. For example, the Vetter Lyo-Ject®, from Vetter Pharma-Fertigung GmbH & Co. KG of Germany is a dual-chambered syringe that is suitable for this purpose. Alternatively, the dual-chambered syringe may be configured such that the gelling initiator 105 is delivered to the cavity first, followed by the polymer 103.

In such embodiments, the polymer and the gelling initiator do, in fact, come into contact with one another as the plunger of the dual-chambered syringe is pushed down, as the barrier between the two chambers is breached. However, as the breaching occurs immediately before delivery of the polymer and the initiator to the cavity, little gelation will have time to occur at the relatively small surface area of the interface between the polymer and the initiator and such) limited gelation should not hamper the delivery (e.g., injection) of either the polymer or the gelling initiator.

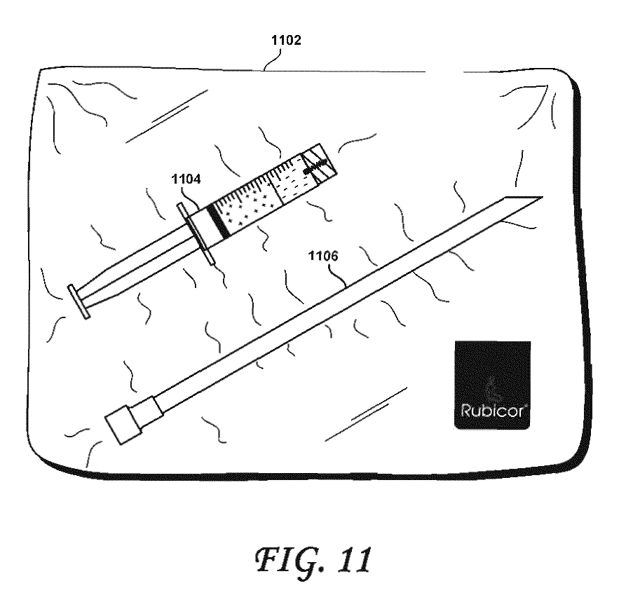

The embodiment of FIG. 10 may be sold as an assembled system comprising a single dual-chambered syringe containing pre-measured amounts of both the polymer 1010 and the initiator and/or initiator agents 1008, with the catheter coupled thereto. Alternatively, such an embodiment is also well suited to kit form, in which the catheter 1106 and the single dual-chambered syringe 1104 are initially decoupled and are packaged in sterile packaging, as suggested at 1102 in FIG. 11. The other embodiments discussed herein may also be readily packaged aid sold in kit form as well.

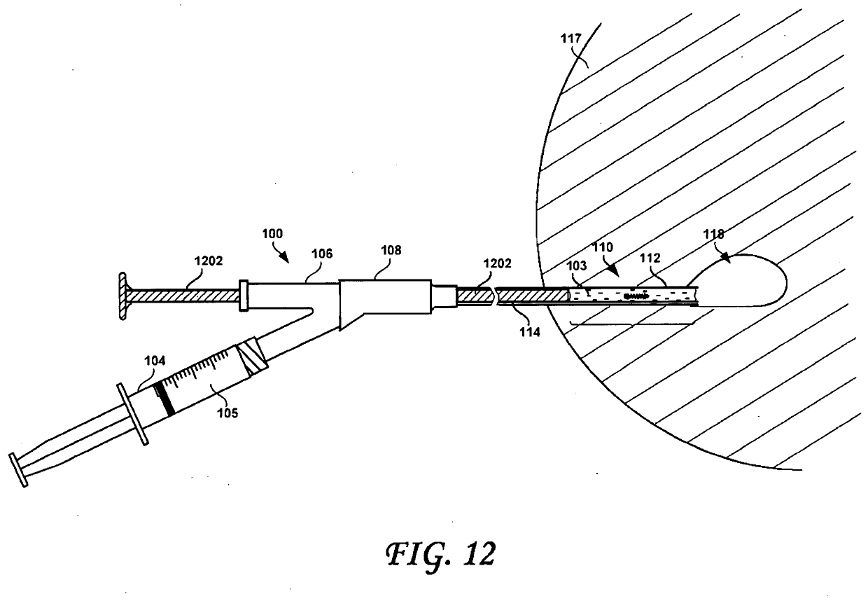

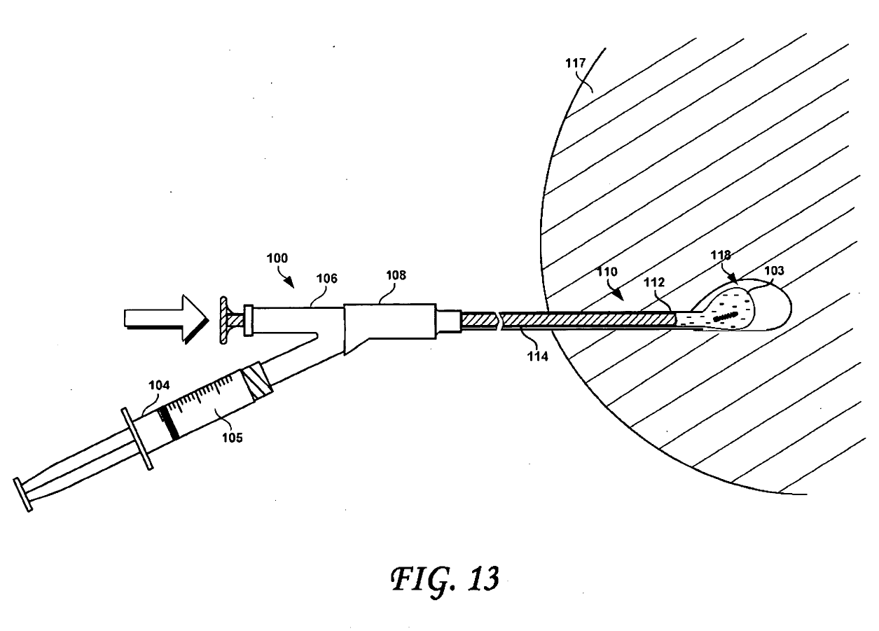

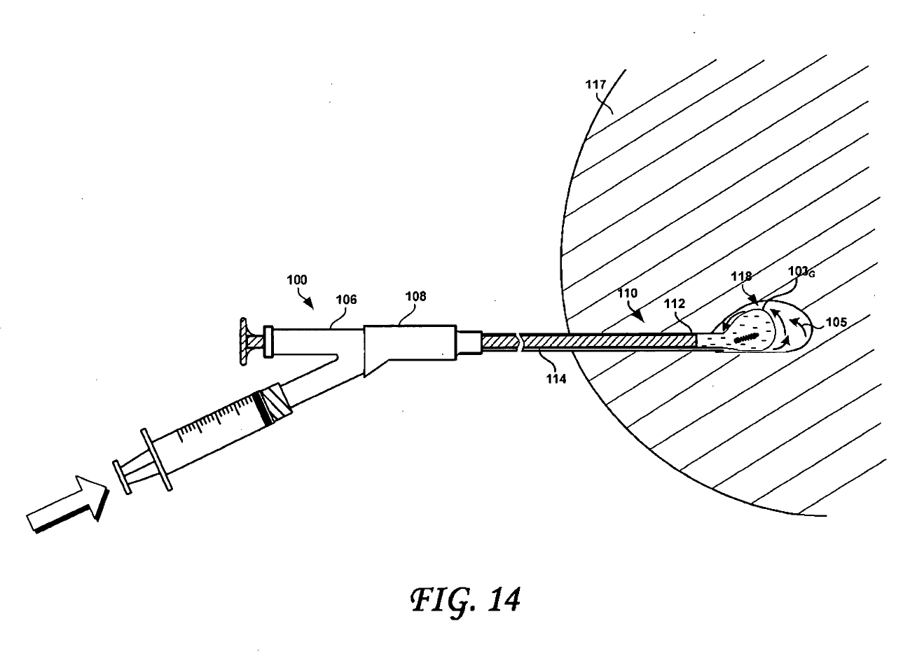

The first source of polymer need not be a syringe. That is, the polymer need not be delivered from a syringe. As shown in FIG. 12, the dual-lumen catheter 112 may be pre-loaded with a measured effective amount of the polymer 103, as well as the radio-opaque element 700A/700B or any other radio-opaque element. The polymer 103 may be pre-loaded in the larger first lumen 116. To push the polymer 103 from the first lumen 116 to the cavity 118, a rod-like piston 1202 may be provided through the “Y” tubing member 106 and the connector 108 and engaged in the first lumen 116 of the dual-lumen catheter 112. The diameter of the piston 1202 may be selected such that the outer surface thereof is in intimate contact with die surface 115 forming the first lumen 116, but still allows the piston 1202 to slide freely in the first lumen 116. This allows the piston 1202 to efficiently push all or nearly all of the polymer 103 out of the first lumen 115 and into the cavity 118, and avoids a volume of polymer and/or initiator being left in the linen after use. Alternatively, the distal end of the plunger 1202 may be fitted with a rubber disc, like the plunger of a syringe. As shown in FIG. 13, to deliver the polymer 103 to the cavity 118, the piston 1202 may be pushed in the distal direction. This pushes the polymer 103 and the radio-opaque element contained in the first lumen 116 forward through the first lumen 116 and out into the cavity 118. Pre-loading the polymer 103 within the catheter 112 as shown allows a more efficient use of the polymer 103 and enables only that which is needed to be preloaded into the catheter 112. As shown in FIG. 14, the initiator 105 may then be delivered to the cavity 118, in the same manner as discussed above; namely, by depressing the plunger of the syringe acting as the second source 104. This causes the initiator 105 to flow through the second lumen 114 of the dual lumen catheter 114 and into the cavity 118, to cause the gelation of the polymer 103, to create the marker 103_{G} in situ, together with the at least partially entrapped radio-opaque element. Note that the polymer 103 is shown in FIG. 13 as being delivered first. However, the initiator 105 contained within the second source 104 may instead be delivered to the cavity 118 before the polymer 103 is delivered thereto.

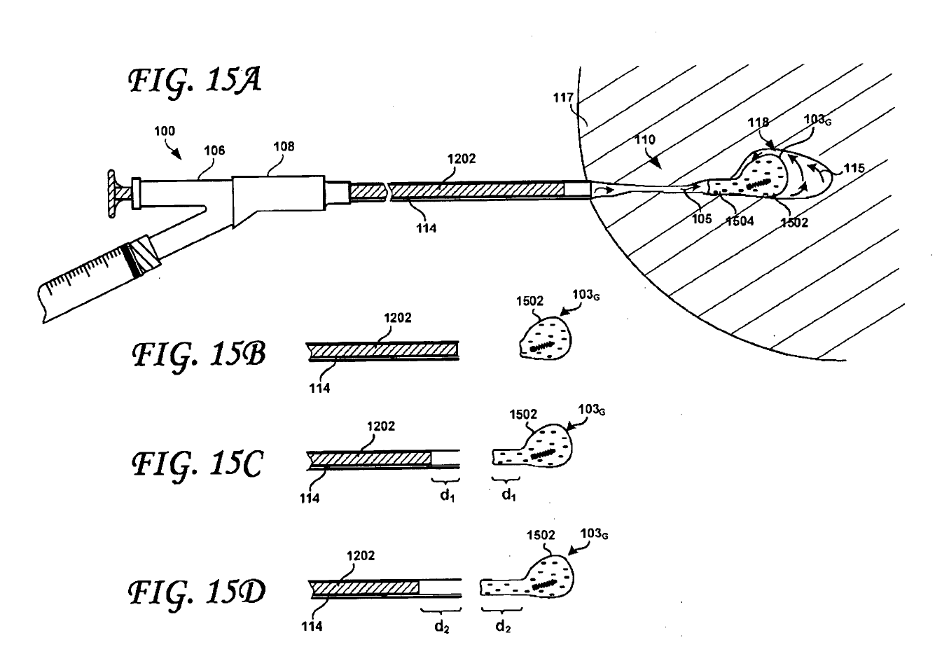

After a few moments to allow for the in situ marker 103_{G} to firm up, the device 100 may then be retracted from the cavity and the patient's tissue, as shown in FIG. 15A. As the initiator 105 is still present within the cavity 118, it will continue to cause the further gelation of the in situ marker, even after the device 100 is retracted from the cavity 118. Note that in FIGS. 12-15A, the distal end of the piston 1202 does not reach the distal end of the catheter 112. Instead, in the embodiment of FIGS. 12-15A, the distal-most reach of the piston 1202 is offset a predetermined distance from the distal tip of the catheter 112. In turn, this causes a portion of the pre-loaded polymer 103 remain in the catheter 112, as shown in FIG. 14. Thereafter, when the device 100 is retracted, a tail portion 1504 remains, in the general shape of the first lumen 116. The length of this tail 1504 is dependent upon the offset of the distal-most tip of the piston 112 from the distal tip of the catheter 112, when the piston 1202 is fully depressed and engaged within the second lumen 116. In this manner, the shape of the in situ marker 103_{G} may be selected by either controlling the length of the piston 1202 and/or the distance the piston 1202 is pushed into the catheter 112. By so doing, the length of the tail 1504 may be controlled. Indeed, as shown in FIG. 15B, the length of the piston 1202 may be selected such that, when fully engaged, the distal tip thereof reaches the distal tip of the catheter 112. This results in little or no tail 1504 being produced, the in situ marker, therefore, including only a bulbous body portion 1502 in which the radio-opaque element is at least partially encapsulated or entrapped. When the piston 1202 is selected such that, when fully engaged into the catheter, the distal tip thereof is offset from the distal tip of the catheter by a distance d₁, a tail (extending from the bulbous body portion 1502) having a length of d₁ will be formed, as shown in FIG. 15C. Alternatively, the physician may choose to not fully depress a piston sized as described relative to FIG. 15B, with similar results. Similarly, a tail having a length of d₂ may be formed by selecting a piston 1202 sized to only reach a distance d₂ from the tip of the catheter 112 or by not fully depressing the piston 1202 within the catheter 112.

The tail 1504 of the iii situ marker is more than a mere artifact of the geometry of the catheter 112 and the piston 1202. The tail 1504 enhances the ability of the marker 103_{G} to become immobilized within the cavity 118 and, significantly, to immobilize the radio-opaque element 700A/700B (or any other radio-opaque element) within the cavity 118. This is because the tail 1504 is engaged within the retraction path of the device 100 and, therefore, functions as an anchor or plug to the in situ marker, anchoring the marker into place and minimizing the likelihood that the in situ marker will migrate within the cavity 118. The functionality of the tail 1504 is increased as the device 100 is retracted from the insertion path thereof, as the act of retracting the device is likely to generate some suction within the insertion/retraction path, which suction will tend to draw the tail 1504 more fully within the insertion retraction path, thereby effectively plugging the insertion/retraction path and further immobilizing the in situ formed marker within the cavity.

The distal tip of the catheter 112 may be blunt, as shown in FIGS. 12-15D, may be tapered or beveled, as shown in FIGS. 9-11, have a more complex shape as shown in FIGS. 1-5 or most any shape that is effective to deliver the polymer 103, the radio-opaque element and the initiator 105 and to shape the resulting in situ marker 103_{G}. The catheter 112, moreover, may be rigid, somewhat or greatly flexible or piece-wise rigid and piece-wise flexible, depending upon the application. It is to be noted that the piston 1202 may provide any needed or desired rigidity to a flexible catheter 112, by imparting its rigidity to a flexible catheter 112 as it is introduced therein. Likewise, the piston 1202 may be fully, somewhat or greatly partially flexible or piece-wise flexible and piece-wise rigid. The piston 1202 should, however, have sufficient column strength to be able to push a more or less viscous polymer 103 through the catheter 112 and out to the cavity 118. The piston may be formed of metal and/or plastic materials to achieve the desired degree of rigidity and/or flexibility. Flexibility of the catheter 112 and/or the piston 1202 may be necessary when using the device 100 in conjunction with other devices, such as the above-described Mammotome® biopsy system. Alternatively still, any desired rigidity may be provided by a separate rigid cannula acting as an introducer. In such as case, the rigid cannula may be inserted into the patient first and the device 100 advanced within the cannula to the cavity 118.

It is to be noted that the embodiments of FIGS. 12-15D need not have the stricture shown therein and that variations are possible. Indeed, a dual lumen catheter 112 may not be needed. Instead, a simple single lumen catheter may be used, thereby obviating the need for at least the “Y” tubing member 106. Indeed, after the piston 1202 has been depressed and the polymer 103 delivered to the cavity 118, the piston may be retracted and removed from the catheter, whereupon a syringe filled with the initiator may be luer-locked onto the catheter and the initiator 105 delivered to the cavity 118. Other variations are possible, as those of skill in this art will appreciate. Indeed, any device(s) or method(s) that are configured to deliver (in whatever order) a radio-opaque element and the precursor constituent elements of the marker (including at least the gelling initiator 105 and the polymer 103) to a natural, preformed or just-created cavity to create all in situ marker are deemed to fall within the scope of the present inventions.

While the foregoing detailed description has described preferred embodiments of the present invention, it is to be understood that the above description is illustrative only and not limiting of the disclosed invention. For example, a single lumen catheter may be configured to deliver a pre-loaded amount of both the polymer 103 and the gelling initiator 105 to the biopsy site. In such an embodiment, the polymer 103 and the gelling initiator may be separated by an (e.g., impermeable) barrier that is configured to be breached or otherwise overcome when the piston is advanced through the catheter. This and the other embodiments shown and described herein may be provided as an assembled system or provided as a kit, packaged in the manner shown and described relative to FIG. 11 or in any other suitable manner. Those of skill in this art may recognize other alternative embodiments and all such alternative embodiments are deemed to fall within the scope of the present invention.

What is claimed is:

1. A soft tissue biopsy cavity marker formed in situ.

2. A method, comprising:
providing a polymer in fluid form;
delivering the provided polymer to a biopsy site;
providing a gelling initiator that is configured to cause the polymer to gel, and
delivering the provided gelling initiator to the biopsy site.

3. The method of claim 2, further comprising a step of providing a radio-opaque element and wherein the first delivering step also delivers the provided radio-opaque element to the biopsy site.

4. The method of claim 2, wherein the first and second delivering steps are carried out such that the polymer and the gelling initiator are initially separated from one another and only come into contact with one another within the biopsy site to form a soft tissue biopsy marker in situ.

5. The method of claim 2, wherein the first providing step is carried out with the polymer being bio-compatible and bio-degradable.

6. The method of claim 2, wherein the first providing step is carried out with the polymer including alginate.

7. The method of claim 2, wherein the second providing step is carried out with the gelling initiator including divalent cations.

8. The method of claim 2, wherein die polymer delivering step is carried out before the gelling initiator delivering step.

9. The method of claim 2, wherein the gelling initiator delivering step is carried out before the polymer delivering step.

10. The method of claim 6, wherein the first providing step is carried out with the alginate being dispersed in an aqueous solution at a concentration of about 0.1% to about 30% by weight.

11. The method of claim 2, wherein the first delivering step delivers about 0.01 cc to about 400 cc of the polymer to the biopsy site and wherein the second delivering step delivers about 0.01 cc to about 800 cc of the initiator to the biopsy site.

12. The method of claim 3, wherein the radio-opaque element providing steps is carried out with the radio-opaque element having a shape that is configured to facilitate the radio-opaque element being entrained with the polymer during the first delivering step.

13. The method of claim 3, wherein the radio-opaque element has a generally cylindrical or helical shape having a first end and a second end, at least the first end being tapered.

14. A device, comprising:
a first source of polymer;
a second source, separate from the first source, of a gelling initiator that is configured to gel the polymer, and
a catheter configured to deliver the polymer and the gelling initiator to a patient.

15. The device of claim 14, wherein the catheter defines a single lumen.

16. The device of claim 14, wherein the catheter defines a first lumen coupled to the first source of the polymer and a second lumen coupled to the second source of the gelling initiator.

17. The device of claim 14, further comprising a radio-opaque element disposed within the first lumen of the dual lumen catheter.

18. The device of claim 14, wherein the polymer includes alginate.

19. The device of claim 14, wherein the gelling initiator includes divalent cations.

20. The device of claim 14, wherein the catheter is configured to deliver the polymer and the gelling initiator separately to the biopsy site.

21. The device of claim 14, wherein the catheter defines an internal lumen and wherein the first source of the polymer is the internal lumen of the catheter.

22. The device of claim 21, further including an elongate piston configured to engage and slide within the internal lumen and to push the polymer out of the internal lumen.

23. The device of claim 22, wherein the piston and the catheter define respective distal tips and wherein the distal tip of the piston is proximal to the distal tip of the catheter when the piston is fully engaged within the catheter.

24. The device of claim 22, wherein at least portions of both the catheter and the piston are configured with selective rigidity and/or flexibility.

25. A kit, comprising:
a device, comprising:
a catheter that is pre-loaded with a bio-compatible and bio-degradable polymer;
a source of a gelling initiator that is configured to gel the polymer, the source of gelling initiator being configured to be coupled to the catheter, and
a radio-opaque element, and
sterile packaging encapsulating the device.

26. The kit of claim 25, wherein the device is formed of plastic materials and is for single use.

27. A breast biopsy marker formed of a gelled polymer, the marker including a bulbous body portion and a tail portion extending from the bulbous body portion.
